(19) United States
(12) Patent Application Publication
(10) Pub. No.: US 2026/0144523 A1
(43) Pub. Date: May 28, 2026

(54) ULTRASOUND DATA PROCESSING SYSTEM, TRANSMISSION CONTROL APPARATUS, PROCESSING APPARATUS, TRANSMISSION CONTROL PROGRAM, AND PROCESSING PROGRAM

(71) Applicant: FUJIFILM Corporation, Tokyo (JP)

(72) Inventors: Hidenao KUBOTA, Tokyo (JP); Tomoaki CHONO, Tokyo (JP)

(73) Assignee: FUJIFILM Corporation, Tokyo (JP)

(21) Appl. No.: 19/398,050

(22) Filed: Nov. 24, 2025

(30) Foreign Application Priority Data
Nov. 26, 2024 (JP) 2024-205645

(51) Int. Cl. A61B 8/00; G06T 7/00

(52) U.S. Cl. CPC A61B 8/5207; A61B 8/54; G06T 7/0012; G06T 2207/10132

(57) ABSTRACT

An ultrasound data processing system includes a transmission control apparatus and a processing apparatus. A first controller of the transmission control apparatus transmits, in a case in which comprehensive data including ultrasound data and processing-related data in association with each other is changed after the comprehensive data is transmitted to the processing apparatus, the changed comprehensive data to the processing apparatus. A second controller of the processing apparatus requests, in a case in which there is specific processing executable by the transmission control apparatus but not executable by the processing apparatus, the transmission control apparatus to provide processing result data indicating a result of the specific processing on the ultrasound data.

### CROSS-REFERENCE TO RELATED APPLICATION

This application claims priority under 35 USC 119 from Japanese Patent Application No. 2024-205645, filed on Nov. 26, 2024, the disclosure of which is incorporated by reference herein.

### BACKGROUND OF THE INVENTION

### 1. Field of the Invention

The present specification discloses improvement of an ultrasound data processing system, a transmission control apparatus, a processing apparatus, a transmission control program, and a processing program.

### 2. Description of the Related Art

In the related art, an ultrasound diagnostic apparatus that forms ultrasound data representing the inside of a subject based on reflected waves from the subject in a case in which ultrasonic waves are transmitted to the subject is known. In addition, in the related art, an ultrasound image processing apparatus that processes the ultrasound data formed by the ultrasound diagnostic apparatus is known. Examples of the ultrasound image processing apparatus include an ultrasound diagnostic apparatus itself and an image analysis apparatus that analyzes or manages the ultrasound data.

In particular, the ultrasound image processing apparatus that comprises various processing modules for executing various types of processing on the ultrasound data, selects a processing module to be used depending on an intended use, and causes the selected processing module to execute processing on the ultrasound data is known. In the present specification, the processing module means hardware alone or a combination of hardware and software for achieving a cohesive function.

For example, JP2003-271924A discloses a medical image processing apparatus that comprises a template in which a procedure of processing medical image data including ultrasound data is described, and a processing module for processing the medical image data, and that processes the medical image data by calling the processing module in accordance with the procedure described in the template.

### SUMMARY OF THE INVENTION

Meanwhile, an ultrasound data processing system including a plurality of ultrasound image processing apparatuses that process the ultrasound data is considered.

In the present specification, the term “ultrasound data” encompasses data obtained by transmitting and receiving the ultrasonic waves to and from the subject, reception beam data before being image-processed, or an ultrasound image formed based on the reception beam data. In addition, the ultrasound data may be two-dimensional (2D) ultrasound data or three-dimensional (3D) ultrasound data in which data indicating the signal intensity of the reflected wave from the subject is arranged in two or three dimensions, and may further be four-dimensional (4D) ultrasound data in which the 3D ultrasound data is arranged in a time direction. In addition, in the present specification, an ultrasound image processing apparatus that is included in the ultrasound data processing system and that transmits the ultrasound data to another ultrasound image processing apparatus will be referred to as a “transmission control apparatus” (a first apparatus according to an embodiment of the present invention), and one or a plurality of ultrasound image processing apparatuses that receive the ultrasound data from the transmission control apparatus and that process the ultrasound data will be referred to as a “processing apparatus” (a second apparatus according to an embodiment of the present invention).

It is considered to manage (store) processing-related data, which is data related to processing on the ultrasound data, in association with the ultrasound data. The processing-related data is, for example, a processing parameter for processing the ultrasound data or processing result data indicating a processing result for the ultrasound data. In the present specification, a combination of the ultrasound data and the processing-related data will be referred to as “comprehensive data”. By transmitting the comprehensive data from the transmission control apparatus to the processing apparatus and storing the comprehensive data related to the same ultrasound data in the transmission control apparatus and the processing apparatus, various benefits can be provided to a user of the ultrasound data processing system. For example, the user of the ultrasound data processing system can execute the same processing on the ultrasound data using the same processing parameter in either the transmission control apparatus or the processing apparatus. In addition, the user of the ultrasound data processing system can confirm the same processing result on the same ultrasound data in either the transmission control apparatus or the processing apparatus.

Here, a case will be considered in which the content of the comprehensive data related to the ultrasound data stored in the transmission control apparatus and the content of the comprehensive data related to the same ultrasound data stored in the processing apparatus are different from each other. For example, the comprehensive data is modified in at least one of the transmission control apparatus or the processing apparatus after the transmission control apparatus transmits the comprehensive data to the processing apparatus. The modification of the comprehensive data may include, for example, an aspect in which the ultrasound data itself is changed (corrected), an aspect in which the processing parameter is updated, an aspect in which the processing result data is corrected, and the like. As described above, in a case in which the content of the comprehensive data related to the ultrasound data stored in the transmission control apparatus and the content of the comprehensive data related to the same ultrasound data stored in the processing apparatus are different from each other, the user of the ultrasound data processing system cannot obtain the above-described benefits.

In addition, the processing that can be executed on the ultrasound data may be different between the transmission control apparatus and the processing apparatus. Specifically, there may be the processing executable by the transmission control apparatus but not executable by the processing apparatus. In this case, even in a case in which the ultrasound data is transmitted from the transmission control apparatus to the processing apparatus, the processing apparatus cannot execute the same processing on the ultrasound data as the processing by the transmission control apparatus.

An object of the ultrasound data processing system disclosed in the present specification is to prevent the content of the comprehensive data related to the same ultrasound data stored in the transmission control apparatus and the content of the comprehensive data related to the same ultrasound data stored in the processing apparatus from being different from each other. Alternatively, an object of the ultrasound data processing system disclosed in the present specification is to enable the processing apparatus to obtain the processing result similar to the processing result obtained by the transmission control apparatus for the ultrasound data, even in a case in which there is processing that is executable by the transmission control apparatus but not executable by the processing apparatus.

The present specification discloses an ultrasound data processing system comprising: a transmission control apparatus including a first processor and a memory; and a processing apparatus including a second processor, in which the memory stores comprehensive data including ultrasound data acquired by transmitting and receiving ultrasonic waves to and from a subject and processing-related data related to processing on the ultrasound data, the first processor is configured to: be capable of executing processing on the ultrasound data; transmit the comprehensive data to the processing apparatus; and transmit, in a case in which the comprehensive data is changed in the transmission control apparatus after the comprehensive data is transmitted to the processing apparatus, the changed comprehensive data to the processing apparatus, and the second processor is configured to: be capable of executing processing on the ultrasound data; specify content of the processing executable by the first processor and content of the processing executable by the second processor; and request, in a case in which there is specific processing that is processing executable by the first processor but not executable by the second processor, the transmission control apparatus to provide processing result data indicating a result of the specific processing on the ultrasound data.

The processing-related data may include at least one of a processing parameter used for the processing on the ultrasound data or processing result data indicating a result of the processing on the ultrasound data.

The first processor may be configured to: be capable of executing a plurality of pieces of processing with different content, including the specific processing; execute, prior to the request for the processing result data from the second processor, the plurality of pieces of processing on the ultrasound data to include, in the comprehensive data, a plurality of processing result data indicating a result of each processing; and transmit, in response to the request for the processing result data from the second processor, the processing result data, which indicates the result of the specific processing and is included in the comprehensive data, to the processing apparatus.

The first processor may be configured to notify, in a case in which the comprehensive data is changed, the processing apparatus that the comprehensive data is changed.

The second processor may be configured to notify, in response to the notification from the first processor, a user of the processing apparatus that the comprehensive data is changed.

The ultrasound data may be four-dimensional ultrasound data representing a temporal change in ultrasound volume data.

The present specification further discloses a transmission control apparatus comprising: a first processor; and a memory, in which the memory stores comprehensive data including ultrasound data acquired by transmitting and receiving ultrasonic waves to and from a subject and processing-related data related to processing on the ultrasound data, and the first processor is configured to: be capable of executing processing on the ultrasound data; transmit the comprehensive data to a processing apparatus capable of executing processing on the ultrasound data; and transmit, in a case in which the comprehensive data is changed in the transmission control apparatus after the comprehensive data is transmitted to the processing apparatus, the changed comprehensive data to the processing apparatus.

The present specification further discloses a processing apparatus comprising: a second processor, in which the second processor is configured to: receive ultrasound data acquired by transmitting and receiving ultrasonic waves to and from a subject from a transmission control apparatus capable of executing processing on the ultrasound data; be capable of executing processing on the ultrasound data; specify content of the processing executable by the transmission control apparatus and content of the processing executable by the second processor; and request, in a case in which there is specific processing that is processing executable by the transmission control apparatus but not executable by the second processor, the transmission control apparatus to provide processing result data indicating a result of the specific processing on the ultrasound data.

The present specification further discloses a transmission control program causing a computer including a memory that stores comprehensive data including ultrasound data acquired by transmitting and receiving ultrasonic waves to and from a subject and processing-related data related to processing on the ultrasound data, to: execute processing on the ultrasound data; transmit the comprehensive data to a processing apparatus capable of executing processing on the ultrasound data; and transmit, in a case in which the comprehensive data is changed in the computer after the comprehensive data is transmitted to the processing apparatus, the changed comprehensive data to the processing apparatus.

The present specification further discloses a processing program causing a computer to: receive ultrasound data acquired by transmitting and receiving ultrasonic waves to and from a subject from a transmission control apparatus capable of executing processing on the ultrasound data; execute processing on the ultrasound data; specify content of the processing executable by the transmission control apparatus and content of the processing executable by the computer; and request, in a case in which there is specific processing that is processing executable by the transmission control apparatus but not executable by the computer, the transmission control apparatus to provide processing result data indicating a result of the specific processing on the ultrasound data.

With the ultrasound data processing system disclosed in the present specification, it is possible to prevent the content of the comprehensive data related to the ultrasound data stored in the transmission control apparatus and the content of the comprehensive data related to the same ultrasound data stored in the processing apparatus from being different from each other. Alternatively, with the ultrasound data processing system disclosed in the present specification, it is possible to enable the processing apparatus to obtain the processing result similar to the processing result obtained by the transmission control apparatus for the ultrasound data, even in a case in which there is processing that is executable by the transmission control apparatus but not executable by the processing apparatus.

### BRIEF DESCRIPTION OF THE DRAWINGS

FIG. 1 is a schematic configuration diagram of an ultrasound data processing system according to the present embodiment.

FIG. 2 is a schematic configuration diagram of an ultrasound diagnostic apparatus.

FIG. 3 is a conceptual diagram showing content of comprehensive data.

FIG. 4A is a diagram showing an example of processing result data.

FIG. 4B is a diagram showing an example of corrected processing result data.

FIG. 5 is a schematic configuration diagram of an image analysis apparatus.

FIG. 6 is a flowchart showing a flow of processing by the ultrasound diagnostic apparatus.

FIG. 7 is a flowchart showing a flow of processing by the image analysis apparatus.

### DESCRIPTION OF THE PREFERRED EMBODIMENTS

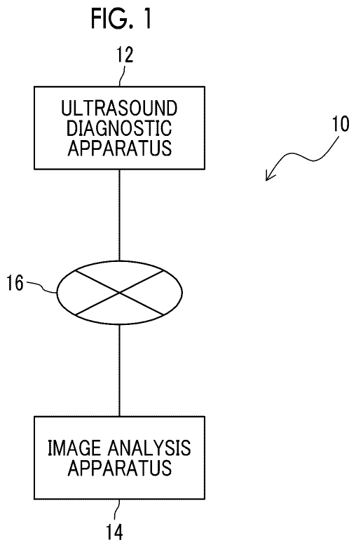

FIG. 1 is a schematic configuration diagram of an ultrasound data processing system 10 according to the present embodiment. The ultrasound data processing system 10 includes an ultrasound diagnostic apparatus 12 as a transmission control apparatus and an image analysis apparatus 14 as a processing apparatus. The example of FIG. 1 shows only one image analysis apparatus 14, but the ultrasound data processing system 10 may include a plurality of image analysis apparatuses 14. The ultrasound diagnostic apparatus 12 and the image analysis apparatus 14 are connected to each other so as to be communicable via a communication line 16 such as a wide area network (WAN) or a local area network (LAN).

Although the details will be described later, an outline of processing executed by the ultrasound data processing system 10 is as follows.

First, ultrasound data is formed in the ultrasound diagnostic apparatus 12. As described above, the term “ultrasound data” encompasses reception beam data and an ultrasound image, and encompasses 2D ultrasound data, 3D ultrasound image data, and 4D ultrasound data. In the present embodiment, the ultrasound data is assumed to be 4D ultrasound data representing a temporal change in 3D ultrasound image data (ultrasound volume data). In addition, the present embodiment is not limited thereto, but in the present embodiment, the ultrasonic waves are transmitted and received to and from the heart of a subject, and the 4D ultrasound data represents the heart.

The ultrasound diagnostic apparatus 12 includes a first processing module that executes processing on the ultrasound data. The first processing module executes processing on the ultrasound data using a processing parameter to acquire processing result data.

The ultrasound diagnostic apparatus 12 stores the ultrasound data and processing-related data related to the processing on the ultrasound data in a memory of the ultrasound diagnostic apparatus 12 in association with each other. That is, the ultrasound diagnostic apparatus 12 stores comprehensive data in the memory. The processing-related data includes at least one of a processing parameter used for the processing on the ultrasound data or processing result data indicating a result of the processing on the ultrasound data. In the present embodiment, the processing parameter and the processing result data are included as the processing-related data. The ultrasound diagnostic apparatus 12 transmits the comprehensive data to the image analysis apparatus 14.

The image analysis apparatus 14 stores the comprehensive data received from the ultrasound diagnostic apparatus 12 in the memory. As a result, a user of the image analysis apparatus 14 can view the comprehensive data (for example, the processing result data). In addition, the image analysis apparatus 14 includes a second processing module that can execute processing equivalent in content to the processing by the first processing module of the ultrasound diagnostic apparatus 12. The second processing module executes the processing on the ultrasound data included in the comprehensive data using the processing parameter included in the comprehensive data to acquire the processing result data.

While the above describes an overview of the processing executed by the ultrasound data processing system 10, in the present embodiment, the ultrasound diagnostic apparatus 12 executes control so that the content of the comprehensive data related to certain ultrasound data stored in the ultrasound diagnostic apparatus 12 and the content of the comprehensive data related to the ultrasound data stored in the image analysis apparatus 14 are not different from each other. In addition, in the present embodiment, even in a case in which there is processing executable by the ultrasound diagnostic apparatus 12 but not executable by the image analysis apparatus 14, the image analysis apparatus 14 is supported to obtain the same processing result as the processing result obtained by the ultrasound diagnostic apparatus 12 for the ultrasound data.

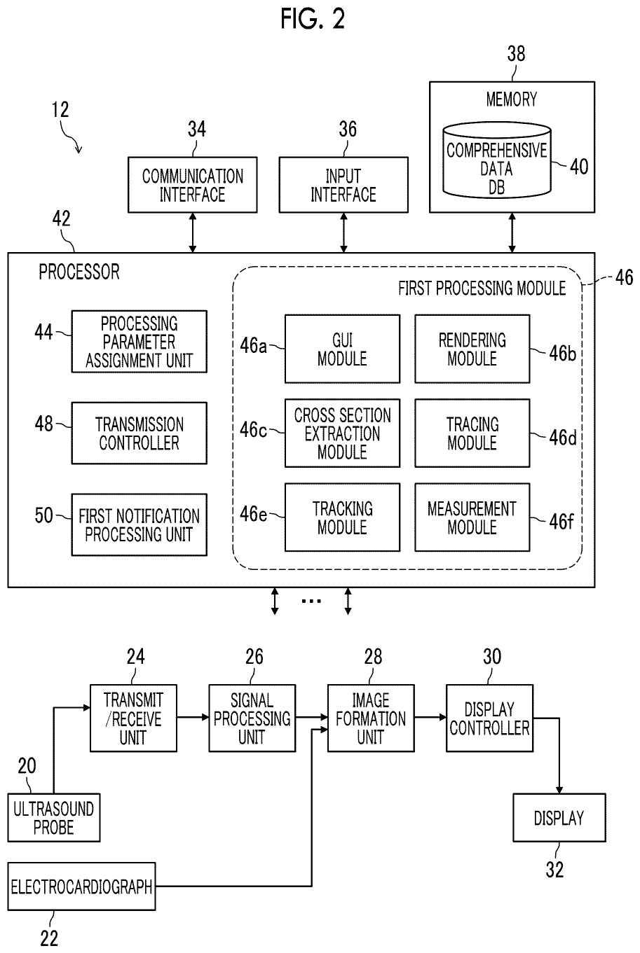

FIG. 2 is a schematic configuration diagram of the ultrasound diagnostic apparatus 12. The ultrasound diagnostic apparatus 12 is a medical apparatus installed in medical institutions such as a hospital.

An ultrasound probe 20 is a device that transmits and receives ultrasonic waves to and from the subject. The ultrasound probe 20 has a transducer element array consisting of a plurality of transducer elements that scan the subject with the ultrasonic beam. The ultrasound probe 20 may be a 1D probe in which a plurality of transducer elements are arranged in one row, or a 2D probe in which a plurality of transducer elements are arranged in a two-dimensional direction. A transmission signal is supplied to each transducer element from a transmit/receive unit 24 described later, so that each transducer element generates the ultrasonic beam that scans an ultrasound scanning plane. Each transducer element receives the reflected waves of the transmitted ultrasonic beam from the subject. In the present embodiment, the ultrasound probe 20 transmits and receives the ultrasonic waves to and from the heart of the subject.

An electrocardiograph 22 detects electrocardiographic data indicating the pulsation of the heart of the subject. For example, the electrocardiograph 22 includes an electrode, the electromotive force of the subject is measured by attaching the electrode to the vicinity of the heart of the subject, and the electrocardiographic data is formed based on the measured electromotive force. The electrocardiographic data detected by the electrocardiograph 22 is transmitted to an image formation unit 28.

The transmit/receive unit 24 transmits the transmission signal to the ultrasound probe 20 (specifically, each transducer element of the transducer element array) under the control of a processor 42 described later. The transmit/receive unit 24 also receives a reception signal from each transducer element that has received the reflected waves from the subject. The transmit/receive unit 24 includes an adder and a plurality of delay elements corresponding to the respective transducer elements, and executes, using the adder and the plurality of delay elements, phase-aligned summation (delay-and-sum) processing in which the reception signals from the respective transducer elements are phase-aligned and added. As a result, reception beam data is formed in which information indicating the signal intensity of the waves reflected from the subject is arranged in a depth direction of the subject.

The signal processing unit 26 executes various types of signal processing including filter processing of applying a bandpass filter, detection processing, and the like, on the reception beam data from the transmit/receive unit 24. As a result, the ultrasound data is obtained. In a case in which the ultrasound probe 20 is a 1D probe, 2D reception beam data (corresponding to one ultrasound tomographic image) are formed as a plurality of reception beam data corresponding to the ultrasound scanning plane. The signal processing unit 26 can form 3D reception beam data (ultrasound volume data) based on a plurality of 2D reception beam data arranged in a direction perpendicular to the ultrasound scanning plane. Furthermore, the signal processing unit 26 can form 4D reception beam data (4D ultrasound volume data) based on a plurality of ultrasound volume data formed over time. In a case in which the ultrasound probe 20 is a 2D probe, the transmit/receive unit 24 forms three-dimensional reception beam data, and thus the signal processing unit 26 can form ultrasound volume data and 4D ultrasound volume data by executing various types of signal processing on the reception beam data. As described above, in the present embodiment, since the ultrasound data is 4D ultrasound data, the signal processing unit 26 forms 4D ultrasound volume data as the 4D ultrasound data.

The image formation unit 28 forms the ultrasound image based on the reception beam data that has been signal-processed by the signal processing unit 26. For example, the image formation unit 28 can form a 2D ultrasound image (ultrasound tomographic image) indicating a cross section (particularly, an ultrasound transmission/reception surface corresponding to the 2D reception beam data) of the subject based on the 2D reception beam data. In addition, the image formation unit 28 forms an electrocardiographic waveform based on the electrocardiographic data received from the electrocardiograph 22. The electrocardiographic waveform is a graph representing the electrical activity of the heart.

A display controller 30 executes control to display various images including the ultrasound tomographic image formed by the image formation unit 28 on a display 32. In addition, the display controller 30 displays the processing result obtained by a first processing module 46, which will be described later, for the ultrasound data on the display 32. In particular, in the present embodiment, since the ultrasound data is the 4D ultrasound data, the display controller 30 may display the electrocardiographic waveform together with a processing result for the 4D ultrasound data (for example, the reconstructed ultrasound image obtained by the rendering processing) and display a phase of a heartbeat corresponding to the processing result.

The display 32 is a display device configured, for example, by a liquid crystal display or an organic electroluminescence (EL) display.

The transmit/receive unit 24, the signal processing unit 26, the image formation unit 28, and the display controller 30 of the ultrasound diagnostic apparatus 12 are implemented by a processor (the processor 42 described later or a processor different from the processor 42).

A communication interface 34 is configured as, for example, a network adapter. The communication interface 34 is configured to communicate with the image analysis apparatus 14 via the communication line 16. In particular, the communication interface 34 transmits the comprehensive data or the like to the image analysis apparatus 14 under the control of a transmission controller 48 described later.

An input interface 36 is configured by, for example, a button, a trackball, and a touch panel. The input interface 36 is used to input an instruction of an operator, who uses the ultrasound diagnostic apparatus 12, to the ultrasound diagnostic apparatus 12.

A memory 38 includes a hard disk drive (HDD), a solid state drive (SSD), an embedded Multi Media Card (eMMC), a read only memory (ROM), a random access memory (RAM), or the like. The memory 38 stores a transmission control program for operating the respective units of the ultrasound diagnostic apparatus 12. The transmission control program can also be stored in a non-transitory computer-readable storage medium such as a Universal Serial Bus (USB) memory or a CD-ROM. The ultrasound diagnostic apparatus 12 can read the transmission control program from such a storage medium and execute the read transmission control program.

In addition, as shown in FIG. 2, a comprehensive data database (DB) 40 is stored in the memory 38. In the comprehensive data DB 40, the ultrasound data (in the present embodiment, 4D ultrasound volume data) formed by the ultrasound diagnostic apparatus 12 and the processing-related data, which is related to the processing on the ultrasound data, are stored in association with each other. As described above, in the present embodiment, as the processing-related data, a processing parameter for processing the ultrasound data and processing result data indicating a result of the processing using the processing parameter on the ultrasound data are stored in association with the ultrasound data. In the present embodiment, the processing parameter to be assigned to the ultrasound data is assigned to (associated with) the ultrasound data by a processing parameter assignment unit 44, which will be described later, but the processing parameter may be assigned to the ultrasound data by another method. For example, the processing parameter may be assigned to the ultrasound data by an apparatus other than the ultrasound diagnostic apparatus 12.

The processor 42 as the first processor functions as the processing parameter assignment unit 44, the first processing module 46, the transmission controller 48, and a first notification processing unit 50 in accordance with the transmission control program stored in the memory 38. Hereinafter, the functions of the respective units that are executed by the processor 42 will be described in detail.

The processing parameter assignment unit 44 assigns one or a plurality of processing parameters to the ultrasound data. The processing parameter assigned to the ultrasound data may be prepared in advance and stored in the memory 38, or the processing parameter assignment unit 44 may calculate the processing parameter or select the processing parameter from among the prepared processing parameter group in accordance with the content of the ultrasound data to assign the processing parameter to the ultrasound data.

As the processing parameter, various parameters required for the first processing module 46 (and a second processing module 76 of the image analysis apparatus 14 described later) to execute the processing on the ultrasound data can be included. In particular, in the present embodiment, since the ultrasound data is 4D ultrasound data, the processing parameters may include a processing parameter for processing the 4D ultrasound data. For example, a case will be considered in which the first processing module 46 executes rendering processing on the 4D ultrasound data to form a reconstructed ultrasound image and displays the reconstructed ultrasound image on the display 32. In this case, as a specific processing parameter for processing the 4D ultrasound data, positional information of a line indicating a cutting plane of a multiplanar reconstruction (MPR) image or a cross section positional information obtained by cropping a rendering image is included.

As a method for assigning the processing parameter to the ultrasound data, several methods can be considered. For example, in a case in which the ultrasound data conforms to the format defined in digital imaging and communications in medicine (DICOM), which is an international standard specification for medical images, the processing parameter assignment unit 44 can record the processing parameter in a data structure defined in DICOM. In the data structure defined in DICOM, each medical data is recorded with a tag number. The tags include a standardized tag (basic information such as a patient name and an examination time) and a private tag that can be freely defined, and the processing parameter assignment unit 44 can record the processing parameter in the private tag. Alternatively, the ultrasound data and the processing parameter may be in separate data files. In this case, the ultrasound data and the processing parameter are associated with each other by another method. For example, by giving the data file of the ultrasound data and the data file of the processing parameter the same name (but with different extensions), the ultrasound data and the processing parameter can be associated with each other.

The first processing module 46 executes the processing on the ultrasound data using the processing parameter assigned to the ultrasound data. In the present embodiment, the first processing module 46 is composed of the GUI module 46a that executes processing related to a graphical user interface (GUI) provided to the operator of the ultrasound diagnostic apparatus 12, the rendering module 46 b that executes rendering processing on 3D or 4D ultrasound data to form a reconstructed ultrasound image that is a 2D ultrasound tomographic image, a cross section extraction module 46c that extracts a desired cross section from the ultrasound data, a tracing module 46d that detects a feature point (feature region) in the ultrasound data, the tracking module 46e that tracks a temporal change in the position of the detected feature point, and the measurement module 46f that executes various types of measurement using the ultrasound data. As described above, the first processing module 46 can execute a plurality of pieces of processing with different content. The processing executable by the first processing module 46 is not limited to these.

The first processing module 46 may be developed by an external developer other than an administrator (for example, a manufacturer) of the ultrasound diagnostic apparatus 12. Further, the first processing module 46 may be customized for the administrator of the ultrasound diagnostic apparatus 12 in response to a request from the administrator.

The first processing module 46 executes the processing on the ultrasound data stored in the comprehensive data DB 40 using the processing parameter assigned to the ultrasound data, based on the instruction from the operator of the ultrasound diagnostic apparatus 12. As a result, the first processing module 46 acquires the processing result data.

The processing result data may indicate a result of sequentially processing the ultrasound data via each processing module included in the first processing module 46.

In addition, as in the present embodiment, in a case in which a plurality of processing modules included in the first processing module 46 can execute a plurality of different pieces of processing on the ultrasound data, a plurality of processing result data indicating a result of each processing module on the ultrasound data may be included in the comprehensive data.

For example, in a case in which the plurality of processing modules included in the first processing module 46 execute a series of processing, the first processing module 46 may execute the processing up to the middle of the series of processing and acquire the processing result data of the processing. Examples of the processing result data include processing result data indicating a result of processing by the rendering module 46b on the ultrasound data, processing result data indicating a result of processing by the rendering module 46b and the cross section extraction module 46c on the ultrasound data, processing result data indicating results of processing by the rendering module 46b, the cross section extraction module 46c, and the tracing module 46d on the ultrasound data, and the like.

In addition, in a case in which the plurality of processing modules included in the first processing module 46 can individually process the unprocessed ultrasound data, the plurality of processing result data indicating the processing result of each processing module included in the first processing module 46 on the unprocessed ultrasound data may be acquired.

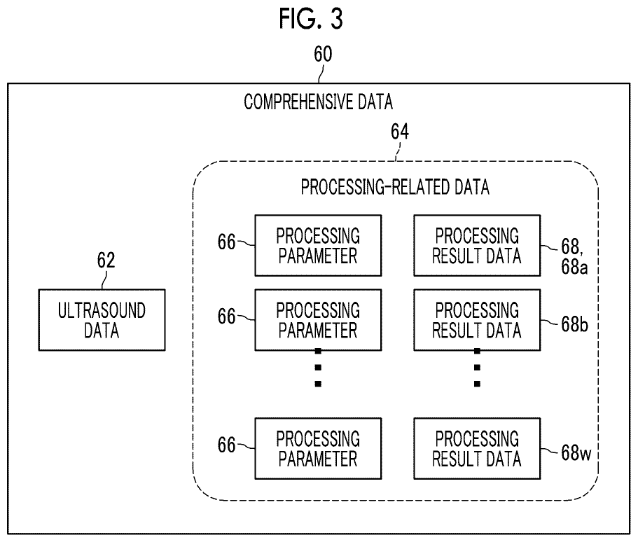

FIG. 3 is a conceptual diagram showing content of the comprehensive data 60. As described above, the comprehensive data 60 is data in which ultrasound data 62 and processing-related data 64 that is related to the processing on the ultrasound data 62 are associated with each other. In the present embodiment, the processing-related data 64 includes one or a plurality of processing parameters 66 for processing the ultrasound data 62 and processing result data 68 that is a result of processing using the processing parameter 66 on the ultrasound data 62.

As described above, the processing parameter assignment unit 44 originally associates the ultrasound data 62 and the processing parameter 66 with each other. The first processing module 46 associates the ultrasound data 62 with the processing result data 68 obtained by the processing of the first processing module 46. For example, by giving the data file of the ultrasound data 62 and the data file of the processing result data 68 the same name (but with different extensions), the ultrasound data 62 and the processing result data 68 can be associated with each other. In the example of FIG. 3, a plurality of processing result data 68 are shown. This is data indicating a result of all processing and a result of processing up to the middle of a series of processing executed on the ultrasound data 62 by the plurality of processing modules included in the first processing module 46. For example, processing result data 68a indicates a result of processing by the rendering module 46b on the ultrasound data 62, processing result data 68b indicates results of processing by the rendering module 46b and the cross section extraction module 46c on the ultrasound data 62, and processing result data 68w indicates a result of processing by the entire first processing module 46 on the ultrasound data 62.

The comprehensive data 60 is stored in the comprehensive data DB 40.

The transmission controller 48 transmits the comprehensive data 60 stored in the comprehensive data DB 40 to the image analysis apparatus 14 based on a transmission request for the comprehensive data 60 received from the image analysis apparatus 14. As a result, the same comprehensive data 60 is stored in both the ultrasound diagnostic apparatus 12 and the image analysis apparatus 14.

After the comprehensive data 60 is transmitted to the image analysis apparatus 14, the comprehensive data 60 may be changed in the ultrasound diagnostic apparatus 12. For example, the ultrasound data 62 itself included in the comprehensive data may be changed (corrected) by the instruction from the operator, automatic calculation, or the like. In addition, the processing parameter 66 included in the comprehensive data may be changed. Further, in a case in which the ultrasound data 62 or the processing parameter 66 included in the comprehensive data is changed, the processing result data 68 may also be changed.

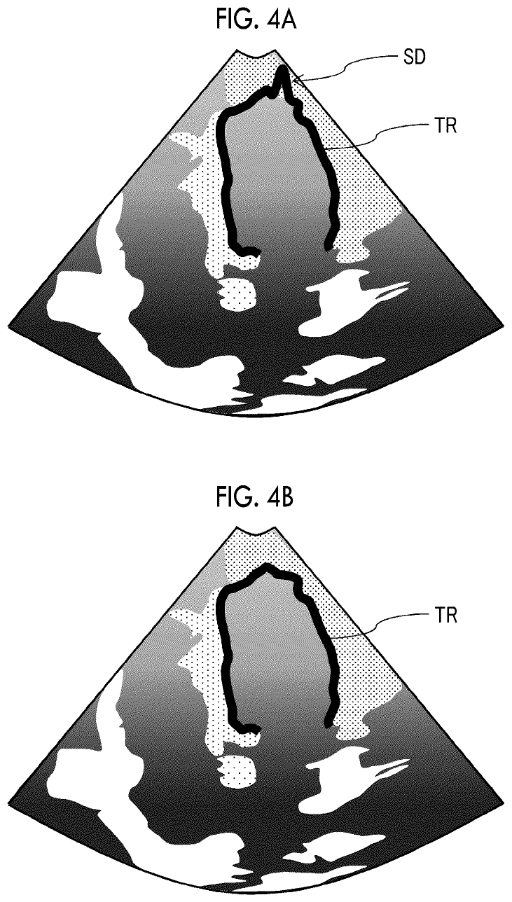

Alternatively, the processing result data 68 may be corrected by the instruction from the operator of the ultrasound diagnostic apparatus 12 or the like. FIG. 4A is a diagram showing the processing result data 68, particularly, a trace TR of a cardiac chamber which is the processing result of the tracing module 46d. The trace TR shown in FIG. 4A includes an error called a shadow SD. The shadow SD does not indicate a true cardiac chamber. The tracing module 46d corrects the trace TR to remove the shadow SD in response to the instruction from the operator input from the input interface 36. FIG. 4B is a diagram showing the corrected processing result data 68, which is the trace TR of the cardiac chamber.

As described above, in a case in which the comprehensive data 60 is changed in the ultrasound diagnostic apparatus 12 after the comprehensive data 60 is transmitted to the image analysis apparatus 14, the comprehensive data 60 stored in the ultrasound diagnostic apparatus 12 and the comprehensive data 60 stored in the image analysis apparatus 14 are different from each other.

Therefore, in a case in which the comprehensive data 60 is changed in the ultrasound diagnostic apparatus 12 after the comprehensive data 60 is transmitted to the image analysis apparatus 14, the transmission controller 48 automatically transmits the changed comprehensive data 60 to the image analysis apparatus 14 without the instruction from the operator of the ultrasound diagnostic apparatus 12. As a result, even in a case in which the comprehensive data 60 is changed in the ultrasound diagnostic apparatus 12 after the comprehensive data 60 is transmitted to the image analysis apparatus 14, the content of the comprehensive data 60 stored in the ultrasound diagnostic apparatus 12 and the content of the comprehensive data 60 stored in the image analysis apparatus 14 can be prevented from being different from each other.

In a case in which the comprehensive data 60 stored in the comprehensive data DB 40 is changed, the first notification processing unit 50 notifies the image analysis apparatus 14 that the comprehensive data 60 is changed. This is to notify the user of the image analysis apparatus 14 that the comprehensive data 60 is changed in the ultrasound diagnostic apparatus 12. It is preferable that this notification also include information (for example, a file name of the comprehensive data (or the ultrasound data 62 included in the comprehensive data 60)) for specifying the changed comprehensive data 60.

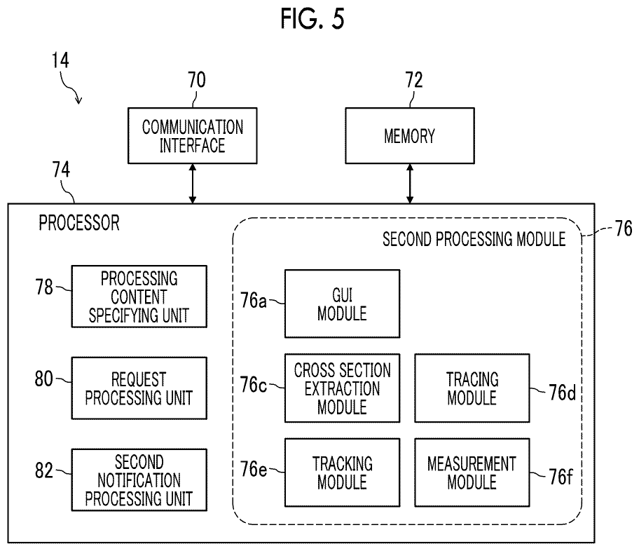

FIG. 5 is a schematic configuration diagram of the image analysis apparatus 14. The image analysis apparatus 14 is an apparatus that provides the image analysis service of analyzing or managing the ultrasound data to a user. The image analysis apparatus 14 may be, for example, a server computer. In the present embodiment, it is assumed that an administrator of the image analysis apparatus 14 is the same as an administrator of the ultrasound diagnostic apparatus 12. However, the administrator of the image analysis apparatus 14 may be different from the administrator of the ultrasound diagnostic apparatus 12 as long as the ultrasound data is shared with the ultrasound diagnostic apparatus 12 and the processing equivalent to the processing by the ultrasound diagnostic apparatus 12 on the ultrasound data is allowed.

For example, the image analysis apparatus 14 is a server computer managed by the administrator of the ultrasound diagnostic apparatus 12, and is a server computer for providing the image analysis service of the same administrator. The image analysis service can also analyze or manage medical image data acquired by various modalities (for example, a computed tomography (CT) apparatus or a magnetic resonance imaging (MRI) apparatus), in addition to the ultrasound data.

A communication interface 70 is configured as, for example, a network adapter. The communication interface 70 is configured to communicate with the ultrasound diagnostic apparatus 12 and a user terminal used by the user of the image analysis service via the communication line 16. In particular, the communication interface 70 receives the comprehensive data 60 from the ultrasound diagnostic apparatus 12. In addition, the communication interface 70 transmits a request (details will be described later) for the processing result data 68 to the ultrasound diagnostic apparatus 12.

The memory 72 includes an HDD, an SSD, an eMMC, a ROM, a RAM, or the like. The memory 72 stores a processing program for operating the respective units of the image analysis apparatus 14. The processing program can also be stored in a non-transitory computer-readable storage medium, such as a USB memory or a CD-ROM. The image analysis apparatus 14 can read the processing program from such a storage medium and execute the read processing program.

The comprehensive data 60 received from the ultrasound diagnostic apparatus 12 is stored in the memory 72. Further, various medical data (including the comprehensive data 60) managed by the image analysis service and the processing result data indicating the result of processing in the image analysis apparatus 14 on the various medical data are stored in the memory 72. In the present specification, processing result data indicating a processing result obtained by the image analysis apparatus 14 (particularly the second processing module 76 described later) for the ultrasound data 62 will be described as “processing-apparatus-side processing result data” in order to distinguish the processing result data from the processing result data 68 indicating the processing result obtained by the ultrasound diagnostic apparatus 12 (particularly the first processing module 46) for the ultrasound data 62.

The processor 74 as the second processor functions as the second processing module 76, a processing content specifying unit 78, a request processing unit 80, and a second notification processing unit 82 in accordance with the processing program stored in the memory 72. Hereinafter, the functions of the respective units that are executed by the processor 74 will be described in detail.

The second processing module 76 executes the processing on the ultrasound data 62 using the processing parameter 66 assigned to the ultrasound data 62, as in the first processing module 46 of the ultrasound diagnostic apparatus 12. In the present embodiment, the second processing module 76 is composed of a GUI module 76a, a cross section extraction module 76c, a tracing module 76d, a tracking module 76e, and a measurement module 76f. The second processing module 76 can also execute a plurality of pieces of processing with different content. The processing executed by the GUI module 76a, the cross section extraction module 76c, the tracing module 76d, the tracking module 76e, and the measurement module 76f is the same as the processing executed by the GUI module 46a, the cross section extraction module 46c, the tracing module 46d, the tracking module 46e, and the measurement module 46f included in the first processing module 46, respectively. That is, the second processing module 76 can execute the processing at least partially equivalent to the processing by the first processing module 46.

In some cases, the first processing module 46 and the second processing module 76 are individually customized. In such a case, the processing module (for example, the cross section extraction module 46c) of the first processing module 46 and the corresponding processing module (for example, the cross section extraction module 76c) of the second processing module 76 may not execute exactly the same processing, but since the bases are the same, the two processing modules execute similar processing. In the present specification, the expression “execute equivalent processing” includes a case in which similar processing is executed. In a case in which the processing is executed on the ultrasound data 62, the first processing module 46 generally obtains better results (with higher performance, higher accuracy, and the like) than the second processing module 76.

In addition, in the comparison between the second processing module 76 and the first processing module 46, there is a processing module that is included in the first processing module 46 but is not included in the second processing module 76. In the examples of FIGS. 2 and 5, the rendering module 46 b corresponds to the above-described module. That is, there is processing executable by the first processing module 46 but not executable by the second processing module 76. In the present specification, such processing will be referred to as “specific processing”.

The reason why the second processing module 76 executes the processing again on the ultrasound data 62 included in the comprehensive data 60 using the processing parameter 66 included in the comprehensive data 60 even though the comprehensive data 60 including the processing result data 68 is received from the ultrasound diagnostic apparatus 12 is that processing results are not always exactly the same between the first processing module 46 and the second processing module 76 as described above. In addition, conversely to the specific processing described above, it is also considered that there is processing executable by the second processing module 76 but not executable by the first processing module 46. In such a case, it is meaningful for the second processing module 76 to execute the processing on the ultrasound data 62 again.

The processing content specifying unit 78 specifies the content of the processing executable by the processor 42 of the ultrasound diagnostic apparatus 12 and the content of the processing executable by the processor 74 of the image analysis apparatus 14. That is, the processing content specifying unit 78 specifies the type of the processing module included in the first processing module 46 and the type of the processing module included in the second processing module 76. Since the second processing module 76 is the function of the processor 74 itself, the processing content specifying unit 78 can naturally specify the type of the processing module included in the second processing module 76. Meanwhile, the type of the processing module included in the first processing module 46 can be specified by various methods. For example, information indicating the type of the processing module included in the first processing module 46 can be received from the ultrasound diagnostic apparatus 12 in advance and stored in the memory 72, and the processing content specifying unit 78 can specify the type of the processing module included in the first processing module 46 by referring to the information. Alternatively, the processing content specifying unit 78 may request the ultrasound diagnostic apparatus 12 to provide the information indicating the type of the processing module included in the first processing module 46.

The request processing unit 80 refers to a result obtained by specifying, via the processing content specifying unit 78, the content of the processing executable by the processor 42 of the ultrasound diagnostic apparatus 12 and the content of the processing executable by the processor 74 of the image analysis apparatus 14, and requests, in a case in which there is processing executable by the processor 42 but not executable by the processor 74 (that is, the specific processing) the ultrasound diagnostic apparatus 12 to provide the processing result data 68 indicating the result of the specific processing on the ultrasound data 62 included in the comprehensive data 60 received from the ultrasound diagnostic apparatus 12.

In the examples of FIGS. 2 and 5, since the processing executed by the rendering module 46b of the first processing module 46 is the specific processing, the request processing unit 80 requests the processing result data 68b indicating the result of the processing by the rendering module 46b on the ultrasound data 62 included in the comprehensive data 60 received from the ultrasound diagnostic apparatus 12. As in the present embodiment, in a case in which the plurality of processing modules included in the first processing module 46 execute the series of processing on the ultrasound data 62, the processing result data 68 indicating the result of the processing up to the specific processing may be requested.

The first processing module 46 of the ultrasound diagnostic apparatus 12 that has received the request transmits the processing result data 68 which indicates the result of the specific processing on the ultrasound data to the image analysis apparatus 14 and is included in the comprehensive data 60 including the ultrasound data 62 for which the request is made. The first processing module 46 may execute the specific processing on the ultrasound data 62 for which the request is made, after receiving the request from the request processing unit 80, and transmit the processing result data 68 indicating the result of the specific processing to the image analysis apparatus 14.

The second processing module 76 of the image analysis apparatus 14 that has received the processing result data 68 of the specific processing executes processing after the specific processing on the ultrasound data 62. Accordingly, even in a case in which the second processing module 76 cannot execute the specific processing, the processing result of the specific processing can be used in the image analysis apparatus 14, and thus the same processing result as the processing result obtained by the ultrasound diagnostic apparatus 12 for the ultrasound data 62 can be obtained in the image analysis apparatus 14.

It is considered that, after the comprehensive data 60 is transmitted from the ultrasound diagnostic apparatus 12 to the image analysis apparatus 14, the comprehensive data may be deleted from the comprehensive data DB 40 of the ultrasound diagnostic apparatus 12. In this case, it is conceivable that the ultrasound diagnostic apparatus 12 does not have the ultrasound data 62 in accordance with the request from the request processing unit 80. In this case, the processor 42 of the ultrasound diagnostic apparatus 12 transmits the request for the ultrasound data 62 to the image analysis apparatus 14. The processor 74 of the image analysis apparatus 14 receives the request and transmits the ultrasound data 62 stored in the memory 72 to the ultrasound diagnostic apparatus 12. The first processing module 46 may execute the specific processing on the received ultrasound data 62 and transmit the processing result data 68 indicating the result to the image analysis apparatus 14.

In addition, in a case in which it is desired to execute as much processing as possible on the second processing module 76 side for the ultrasound data 62, the second processing module 76 may execute the processing up to the processing before the specific processing in the series of processing of the first processing module 46 or the second processing module 76, and the request processing unit 80 may transmit the processing-apparatus-side processing result data obtained in this way to the ultrasound diagnostic apparatus 12 and request the ultrasound diagnostic apparatus 12 to provide the processing result data 68 indicating the result of the specific processing. The first processing module 46 of the ultrasound diagnostic apparatus 12 that has received the processing-apparatus-side processing result data and the request executes the specific processing on the ultrasound data based on the processing result indicated by the processing-apparatus-side processing result data, and transmits the processing result data 68 indicating the result to the image analysis apparatus 14.

The second notification processing unit 82 notifies the user of the image analysis apparatus 14 that the comprehensive data 60 stored in the comprehensive data DB 40 is changed in response to the notification from the first notification processing unit 50 of the ultrasound diagnostic apparatus 12. The second notification processing unit 82 transmits the notification, for example, by displaying a message on a display of the user terminal used by the user of the image analysis service. Accordingly, the user of the image analysis apparatus 14 can easily understand that the comprehensive data 60 stored in the comprehensive data DB 40 is changed.

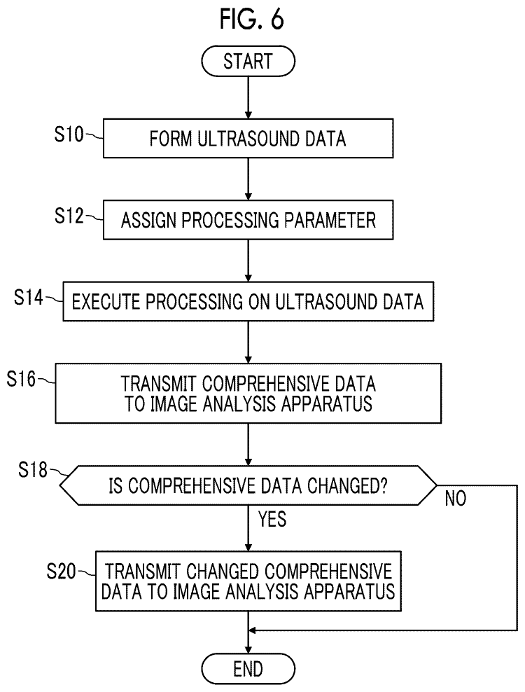

The outline of the ultrasound data processing system 10 according to the present embodiment has been described above. Hereinafter, a flow of the processing by the ultrasound diagnostic apparatus 12 will be described with reference to a flowchart shown in FIG. 6.

In step S10, the signal processing unit 26 forms the ultrasound data 62 based on the reception signal obtained by transmitting and receiving the ultrasonic waves to and from the subject using the ultrasound probe 20.

In step S12, the processing parameter assignment unit 44 assigns the processing parameter 66 to the ultrasound data formed in step S10.

In step S14, the first processing module 46 executes the processing on the ultrasound data 62 formed in step S10 using the processing parameter 66 assigned in step S12. As a result, the first processing module 46 acquires the processing result data 68. The ultrasound data 62, the processing parameter 66, and the processing result data 68 are stored in the comprehensive data DB 40 as the comprehensive data 60 in association with each other.

In step S16, the transmission controller 48 transmits the comprehensive data 60 formed in step S14 to the image analysis apparatus 14. The comprehensive data 60 is stored in the memory 72 of the image analysis apparatus 14.

In step S18, the transmission controller 48 determines whether or not the comprehensive data 60 formed in step S14 is changed in the ultrasound diagnostic apparatus 12. In a case in which there is no change, the processing ends, and in a case in which there is the change, the processing proceeds to step S20.

In step S20, the transmission controller 48 transmits the changed comprehensive data 60 to the image analysis apparatus 14.

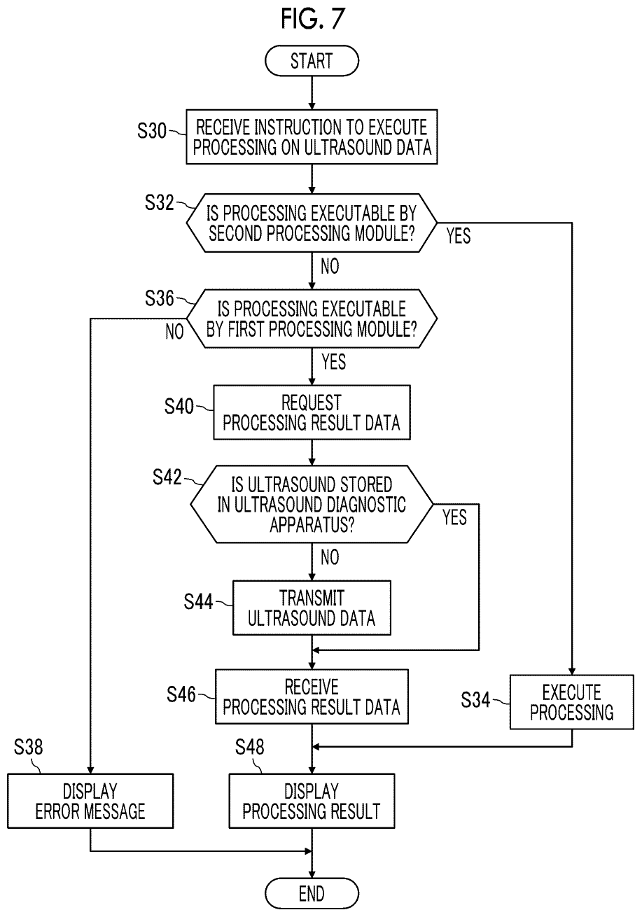

Hereinafter, a flow of processing of the image analysis apparatus 14 will be described with reference to the flowchart shown in FIG. 7. It is assumed that, at the start of the flowchart of FIG. 7, the image analysis apparatus 14 has received the comprehensive data 60 from the ultrasound diagnostic apparatus 12, and the comprehensive data 60 is stored in the memory 72.

In step S30, the image analysis apparatus 14 receives an instruction to execute the processing on the ultrasound data 62 from the user of the image analysis service provided by the image analysis apparatus 14.

In step S32, the processing content specifying unit 78 determines whether or not the second processing module 76 can execute the processing related to the execution instruction received in step S30. In a case in which the second processing module 76 can execute the processing, the processing proceeds to step S34, and in step S34, the second processing module 76 executes the processing related to the execution instruction on the ultrasound data 62 and acquires the processing-apparatus-side processing result data.

In a case in which the second processing module 76 cannot execute the processing related to the execution instruction received in step S30, the processing proceeds to step S36. In step S36, the processing content specifying unit 78 determines whether or not the first processing module 46 of the ultrasound diagnostic apparatus 12 can execute the processing related to the execution instruction received in step S30. In a case in which the first processing module 46 cannot execute the processing related to the execution instruction received in step S30, that is, in a case in which the first processing module 46 and the second processing module 76 cannot execute the processing, the processing proceeds to step S38.

In step S38, the second notification processing unit 82 displays an error message on the display of the user terminal used by the user of the image analysis service. The error message is a message indicating that the processing related to the execution instruction from the user cannot be executed by the ultrasound data processing system 10.

In a case in which the first processing module 46 can execute the processing related to the execution instruction received in step S30, that is, in a case in which the first processing module 46 can execute the specific processing, the processing proceeds from step S36 to step S40. In step S40, the request processing unit 80 requests the ultrasound diagnostic apparatus 12 to provide the processing result data 68 indicating the result of the specific processing on the ultrasound data 62 related to the execution instruction received in step S30.

In step S42, the request processing unit 80 determines whether or not the ultrasound data 62 in accordance with the request is stored in the ultrasound diagnostic apparatus 12. As described above, in a case in which the ultrasound data 62 in accordance with the request is not stored in the ultrasound diagnostic apparatus 12, the processor 42 of the ultrasound diagnostic apparatus 12 transmits the request for the ultrasound data 62 to the image analysis apparatus 14. The request processing unit 80 can determine that the ultrasound data 62 in accordance with the request is not stored in the ultrasound diagnostic apparatus 12 in a case in which the request for the ultrasound data 62 is received from the ultrasound diagnostic apparatus 12. In a case in which the ultrasound data 62 in accordance with the request is not stored in the ultrasound diagnostic apparatus 12, the processing proceeds to step S44, and in a case in which the ultrasound data 62 in accordance with the request is stored in the ultrasound diagnostic apparatus 12, step S44 is bypassed, and the processing proceeds to step S46.

In step S44, the processor 74 of the image analysis apparatus 14 receives the request and transmits the ultrasound data 62 stored in the memory 72 to the ultrasound diagnostic apparatus 12. The first processing module 46 of the ultrasound diagnostic apparatus 12 executes the specific processing on the received ultrasound data 62 to obtain the processing result data.

In step S46, the transmission controller 48 of the ultrasound diagnostic apparatus 12 transmits the processing result data 68 in accordance with the request in step S40 to the image analysis apparatus 14. The image analysis apparatus 14 receives the processing result data 68.

In step S48, the processor 74 of the image analysis apparatus 14 displays the received processing result data 68 on the display of the user terminal used by the user of the image analysis service. Alternatively, the second processing module 76 may further execute processing on the ultrasound data 62 based on the received processing result data 68 and display the processing-apparatus-side processing result data on the display of the user terminal.

Although the ultrasound data processing system according to the present disclosure has been described above, the ultrasound data processing system according to the present disclosure is not limited to the above-described embodiment, and various changes can be made without departing from the gist of the present disclosure.

For example, in the above-described embodiment, the transmission control apparatus is the ultrasound diagnostic apparatus 12, but the transmission control apparatus may be any computer as long as the comprehensive data DB 40 that stores the comprehensive data 60 is included, and the first processing module 46 and the transmission controller 48 are included. In addition, the function of the transmission control apparatus may be exhibited by cooperation of a plurality of apparatuses. In this case, the plurality of apparatuses correspond to the transmission control apparatus. Similarly, in the above-described embodiment, the processing apparatus is the image analysis apparatus 14, but the processing apparatus may be any computer as long as the comprehensive data 60 is received from the transmission control apparatus, and the second processing module 76, the processing content specifying unit 78, and the request processing unit 80 are included. In addition, the function of the processing apparatus may be exhibited by cooperation of a plurality of apparatuses. In this case, the plurality of apparatuses correspond to the processing apparatus.

In the present embodiment, each type of processing is executed by any computer. In addition, any computer may execute these pieces of processing by a processor as hardware, a program as software, or a combination thereof. In such a case, the processor is configured to execute various pieces of processing in the present embodiment in cooperation with the program, and may function as each unit or each means in the present embodiment. In addition, the execution order of the processing by the processor is not limited to the above-described order and may be changed as appropriate. Any computer may be a general-purpose computer, a computer for specific use, a workstation, or another system that can execute each type of processing.

The processor may be configured by one or more types of hardware, and the type of hardware is not limited. For example, the processor may be implemented by hardware such as a programmable logic device, for example, a central processing unit (CPU), a micro processing unit (MPU), or a field programmable gate array (FPGA), a dedicated circuit for executing specific processing such as an application specific integrated circuit (ASIC), a graphics processing unit (GPU), or a neural processing unit (NPU). Moreover, the kind of hardware may be a combination of different types of hardware. In a case in which the plurality of types of hardware are configured to execute one or a plurality of pieces of processing of a certain processor, the plurality of types of hardware may be present in devices physically separated from each other or may be present in the same device. Furthermore, in any of the embodiments, the order of each type of processing executed by the processor is not limited to the above-described order, and may be changed as appropriate. In addition, hardware is implemented in a form of an electric circuit (circuitry) in which circuit elements, such as semiconductor elements, are combined.

Furthermore, the program may be software such as firmware or microcode. The program may be, for example, a group of program modules, and each function thereof may be implemented by a processor configured to execute each function. The program may be a program code or a plurality of code segments stored in one or more non-transitory computer-readable media (for example, a storage medium and other storages). The program may be stored in the plurality of non-transitory computer-readable media present in devices physically separated from each other. The program code or the code segment may represent any combination of procedures, functions, subprograms, routines, subroutines, modules, software packages, classes, instructions, data structures, or program statements. The program code or the code segment may be connected to another code segment or a hardware circuit by transmitting and receiving information, data, arguments, parameters, or contents in the memory.

The present invention is also applicable to a program and a program product.

What is claimed is:

1. An ultrasound data processing system comprising:
a transmission control apparatus including a first processor and a memory; and
a processing apparatus including a second processor,
wherein the memory stores comprehensive data including ultrasound data acquired by transmitting and receiving ultrasonic waves to and from a subject and processing-related data related to processing on the ultrasound data,
the first processor is configured to:
be capable of executing processing on the ultrasound data;
transmit the comprehensive data to the processing apparatus; and
transmit, in a case in which the comprehensive data is changed in the transmission control apparatus after the comprehensive data is transmitted to the processing apparatus, the changed comprehensive data to the processing apparatus, and
the second processor is configured to:
be capable of executing processing on the ultrasound data;
specify content of the processing executable by the first processor and content of the processing executable by the second processor; and
request, in a case in which there is specific processing that is processing executable by the first processor but not executable by the second processor, the transmission control apparatus to provide processing result data indicating a result of the specific processing on the ultrasound data.

2. The ultrasound data processing system according to claim 1,
wherein the processing-related data includes at least one of a processing parameter used for the processing on the ultrasound data or processing result data indicating a result of the processing on the ultrasound data.

3. The ultrasound data processing system according to claim 1,
wherein the first processor is configured to:
be capable of executing a plurality of pieces of processing with different content, including the specific processing;
execute, prior to the request for the processing result data from the second processor, the plurality of pieces of processing on the ultrasound data to include, in the comprehensive data, a plurality of processing result data indicating a result of each processing; and
transmit, in response to the request for the processing result data from the second processor, the processing result data, which indicates the result of the specific processing and is included in the comprehensive data, to the processing apparatus.

4. The ultrasound data processing system according to claim 1,
wherein the first processor is configured to notify, in a case in which the comprehensive data is changed, the processing apparatus that the comprehensive data is changed.

5. The ultrasound data processing system according to claim 4,
wherein the second processor is configured to notify, in response to the notification from the first processor, a user of the processing apparatus that the comprehensive data is changed.

6. The ultrasound data processing system according to claim 1,
wherein the ultrasound data is four-dimensional ultrasound data representing a temporal change in ultrasound volume data.

7. A transmission control apparatus comprising:
a first processor; and
a memory,
wherein the memory stores comprehensive data including ultrasound data acquired by transmitting and receiving ultrasonic waves to and from a subject and processing-related data related to processing on the ultrasound data, and
the first processor is configured to:
be capable of executing processing on the ultrasound data;
transmit the comprehensive data to a processing apparatus capable of executing processing on the ultrasound data; and
transmit, in a case in which the comprehensive data is changed in the transmission control apparatus after the comprehensive data is transmitted to the processing apparatus, the changed comprehensive data to the processing apparatus.

8. A processing apparatus comprising:
a second processor,
wherein the second processor is configured to:
receive ultrasound data acquired by transmitting and receiving ultrasonic waves to and from a subject from a transmission control apparatus capable of executing processing on the ultrasound data;
be capable of executing processing on the ultrasound data;
specify content of the processing executable by the transmission control apparatus and content of the processing executable by the second processor; and
request, in a case in which there is specific processing that is processing executable by the transmission control apparatus but not executable by the second processor, the transmission control apparatus to provide processing result data indicating a result of the specific processing on the ultrasound data.
